(19) United States
(12) Patent Application Publication
(10) Pub. No.: US 2025/0170157 A1
(43) Pub. Date: May 29, 2025

(54) METHODS AND COMPOSITIONS FOR THE TREATMENT OF HEPATIC ENCEPHALOPATHY

(71) Applicant: LEXICON PHARMACEUTICALS, INC., The Woodlands, TX (US)

(72) Inventor: Christopher Wilder Warner, Pittsboro, NC (US)

(21) Appl. No.: 18/939,816

(22) Filed: Nov. 7, 2024

Related U.S. Application Data
(60) Provisional application No. 63/597,900, filed on Nov. 10, 2023.

(51) Int. Cl. A61K 31/7016; A61P 25/00

(52) U.S. Cl. CPC A61K 31/7016; A61P 25/00

(57) ABSTRACT

Methods of treating hepatic encephalopathy and compounds and compositions useful therein are disclosed.

This application claims priority to U.S. provisional patent application No. 63/597,900, filed Nov. 10, 2023, the entirety of which is incorporated herein by reference.

### FIELD OF THE INVENTION

This invention relates to methods of treating hepatic encephalopathy and compounds and compositions useful therein.

### BACKGROUND

Hepatic encephalopathy (HE) is broadly defined as brain dysfunction caused by liver insufficiency and/or portal-systemic shunting, which manifests as a wide spectrum of neurological or psychiatric abnormalities ranging from subclinical alterations to coma. Rose, C. F., et al., Hepatic encephalopathy: Novel insights into classification, pathophysiology and therapy, Journal of Hepatology 2020 vol. 73:1526-1547, 1526. Characterized predominantly by alterations of personality, consciousness, cognition, and motor function, HE is “probably the most frequent complication of cirrhosis that leads to hospitalizations and repeated re-admissions.” Id.

Three types of HE are traditionally differentiated according to the underlying cause: Type A as an essential component of acute liver failure, type B as a consequence of porto-systemic shunts in the absence of liver dysfunction and type C in patients with liver cirrhosis and porto-systemic bypass. Weissenborn, K., Hepatic Encephalopathy: Definition, Clinical Grading and Diagnostic Principles, Drugs (2019) 79 (Suppl 1):S5-S9, S5. Type C typically occurs in response to well-recognized precipitating factors, which include infection, gastrointestinal bleeding, diuretic overdose, electrolyte disorder, constipation, psychoactive medication, dehydration, and dietary indiscretion. Id. at S5.

The West Haven Criteria (WHC) are frequently used for grading HE. This grading system differentiates five grades of HE. In grade I, patients show a lack of attention and some subtle personality changes that are obvious predominantly to their relatives. In grade II, patients experience a distortion of time in combination with inappropriate behavior and lethargy. In grade III, patients are stuporous but respond to stimuli. They are also disoriented for place and situation and may exhibit bizarre behavior. In grade IV, patients are in coma. The fifth grade, referred to as subclinical or minimal HE (MHE), encompasses patients without clinical signs of HE but who exhibit alterations of brain function in neuropsychological or neurophysiological measures. Id. at S6.

Although HE typically manifests as alterations of consciousness and cognitive dysfunction, it may also manifest as motor symptoms, which include extrapyramidal and cerebellar signs, such as hypomimia, bradykinesia, rigidity, tremor, dysarthria, ataxia, and flapping tremor (asterixis), which may be the most often cited motor symptom of HE. Such symptoms are not strictly tied to a distinct grade of HE. Id.

While diagnosis of covert or minimal forms of HE (e.g., WHC grade V) can be difficult, diagnosis of overt HE “is relatively straightforward and usually a diagnosis of exclusion.” Rose, supra, at 1534-1535. For example, a normal ammonia concentration can be used to rule out the diagnosis of overt HE because the negative predictive value of a normal ammonia concentration is high. Id. at 1535. Although ammonia levels cannot alone indicate HE severity, more severe HE is associated with higher levels. Id.

Some existing therapies for HE target ammonia. For example, the non-absorbable disaccharides, lactulose and lactitol, are recommended as first-line treatment for HE. Id. at 1537. Unfortunately, lactulose therapy is associated with adverse effects, such as nausea, cramping, and bloating, that limit its use. Recently, ornithine phenylacetate—a drug combination designed to increase muscle detoxification—gained attention as a potential new treatment option, but a phase 2 clinical trial showed no difference between patients treated with the composition and those treated with placebo. Rahimi, R. S., et al., Efficacy and Safety of Ornithine Phenylacetate for Treating Overt Hepatic Encephalopathy in a Randomized Trial, Clinical Gastroenterology and Hepatology 2021; 19:2626-2635. Other therapies, such as antimicrobial agents, probiotics, branched-chain amino acids, L-omithine L-aspartate (LOLA), and albumin have been used to treat HE, with varying degrees of success. Rose, supra, at 1537-1539. A need for new methods for treating the condition clearly remains.

### SUMMARY

This invention is directed, in part, to methods and compositions for the treatment of hepatic encephalopathy. One embodiment of this invention encompasses a method of treating or preventing hepatic encephalopathy, which method comprises administering to a patient in need thereof a therapeutically or prophylactically effective amount of a compound that inhibits SLGT1 and has minimal systemic exposure.

A preferred compound is N-(1-((2-(dimethylamino)ethyl)-amino)-2-methyl-1-oxopropan-2-yl)-4-(4-(2-methyl-5-((2S,3R,4R,5S,6R)-3,4,5-trihydroxy-6-(methylthio)tetrahydro-2H-pyran-2-yl)benzyl)phenyl)butanamide (LX2761) or a pharmaceutically acceptable salt or co-crystal thereof. In certain embodiments of the invention, the compound is N-(1-((2-(dimethylamino)ethyl)-amino)-2-methyl-1-oxopropan-2-yl)-4-(4-(2-methyl-5-((2S,3R,4R,5S,6R)-3,4,5-trihydroxy-6-(methylthio)tetrahydro-2H-pyran-2-yl)benzyl)phenyl)butanamide L-proline. In particular embodiments, the compound is crystalline.

In certain embodiments of the invention, the patient is an adult human and the therapeutically or prophylactically effective amount is 0.025-1 mg (e.g., 0.25, 0.5, 0.1, 0.2, 0.3, 0.4, 0.5, 0.6 mg). In one embodiment, the compound is administered once daily (qd) or twice daily (bid).

In certain embodiments, the hepatic encephalopathy is WHC grade I-III. In one embodiment, the hepatic encephalopathy is subclinical or minimal hepatic encephalopathy (e.g., WHC grade V).

In certain embodiments, the patient is taking an antibiotic, lactulose/lactitol, LOLA, ornithine phenylacetate, glycerol/sodium phenylbutyrate, or zinc.

In some embodiments, the SGLT1 inhibitor is administered in combination with one or more agents currently used to treat HE. Examples of such agents include: antibiotics (e.g., rifaximin, neomycin/paromomycin/metronidazole, vancomycin); lactulose/lactitol; LOLA (a preparation of L-ornithine and L-aspartate); ornithine phenylacetate; glycerol/sodium phenylbutyrate; and zinc (to correct underlying deficiency common in cirrhotic patients).

This invention also encompasses compositions useful in the treatment, management, and/or prevention of HE. For example, one embodiment of the invention encompasses a composition comprising a compound and a second active agent, wherein the compound is an SGLT1 inhibitor with minimal systemic exposure and the second active agent is an antibiotic, lactulose/lactitol, LOLA, ornithine phenylacetate, glycerol/sodium phenylbutyrate, or zinc. In specific embodiments, the compound is LX2761 or a pharmaceutically acceptable salt or co-crystal thereof.

### BRIEF DESCRIPTION OF THE DRAWINGS

FIGS. 1A-1F present data showing the effect of LX2761 on glycemic control in mice with STZ-induced diabetes. Specifically, FIG. 1A shows oral glucose tolerance test (OGTT) glucose excursions on day 20. Glucose excursions among groups were compared as glucose AUC0-120 minutes values. FIG. 1B shows the change in A1C levels on day 32 relative to baseline values. On day 49, the final study day, samples were obtained to measure FIG. 1C fasting blood glucose; FIG. 1D plasma tGLP-1; and FIG. 1E cecal glucose, reported as the total amount of glucose recovered. FIG. 1F shows the effect of LX2761 on cecal pH (a direct pH measurement of cecal contents).

FIG. 2 provides a table with baseline Bristol Stool Form (BSF) Scale data for subjects taking placebo or LX2761 in a multiple ascending dose study.

FIG. 3 shows the frequency of BSF Scale type 6 or 7 data as a function of time from patients in cohort A2 of the multiple ascending dose study.

### DETAILED DESCRIPTION

This invention is directed to methods of treating, managing, and/or preventing hepatic encephalopathy (HE), and compounds and compositions useful therein.

Unless otherwise indicated, the terms “manage,” “managing” and “management” mean preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder or changing the way that a patient responds to the disease or disorder.

Unless otherwise indicated, the terms “prevent,” “preventing” and “prevention” contemplate an action that occurs before a patient begins to suffer from the specified disease or disorder, which inhibits or reduces the severity of the disease or disorder. The terms encompass prophylaxis.

Unless otherwise indicated, a “prophylactically effective amount” of a compound is an amount sufficient to prevent a disease or condition, or one or more symptoms associated with the disease or condition, or prevent its recurrence. A “prophylactically effective amount” of a compound means an amount, alone or in combination with other agents, that provides a prophylactic benefit in the prevention of the disease. The term “prophylactically effective amount” can encompass an amount that improves overall prophylaxis or enhances the prophylactic efficacy of another prophylactic agent.

Unless otherwise indicated, a “therapeutically effective amount” of a compound is an amount sufficient to provide a therapeutic benefit in the treatment or management of a disease or condition, or to delay or minimize one or more symptoms associated with the disease or condition. A “therapeutically effective amount” of a compound means an amount, alone or in combination with other therapies, that provides a therapeutic benefit in the treatment or management of the disease or condition. The term “therapeutically effective amount” can encompass an amount that improves overall therapy, reduces, or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from a specified disease or disorder, which reduces the severity of the disease or disorder or retards or slows the progression of the disease or disorder.

As used herein, the terms “subject” and “patient” are used interchangeably. The terms “subject” and “subjects” refer to an animal, such as a non-primate mammal (e.g., cow, pig, horse, cat, dog, rat, and mouse) and a primate (e.g., monkey, a chimpanzee, human). Preferred subjects are human (e.g., adult humans).

In general, this invention is directed to methods of treating, managing or preventing HE that comprise administering to a subject (e.g., a human patient) in need thereof a therapeutically or prophylactically effective amount of an SGLT1 inhibitor. Preferred SGLT1 inhibitors have little or no systemic exposure.

As used herein, the term “SGLT1 inhibitor” refers to a compound that inhibits SGLT1 with an IC₅₀ of less than 50, 25 or 10 nM, which can be measured by methods well known in the art. See, e.g., U.S. Pat. No. 10,106,569.

As used herein, the terms “minimal systemic exposure” and “locally-acting” are used interchangeably to refer to a compound that, when orally administered to male C57 mice at a dose of 10 mg/kg, affords a maximum plasma concentration (Cₘₐₓ) of less than 250, 100, 50 or 10 nM. This can be measured by methods well known in the art, such as LC/MS.

Particular SGLT1 inhibitors are disclosed in U.S. Pat. No. 10,106,569. A preferred SGLT1 inhibitor is N-(1-((2-(dimethylamino)ethyl)-amino)-2-methyl-1-oxopropan-2-yl)-4-(4-(2-methyl-5-((2S,3R,4R,5S,6R)-3,4,5-trihydroxy-6-(methylthio)tetrahydro-2H-pyran-2-yl)benzyl)phenyl)butanamide (“LX2761”):

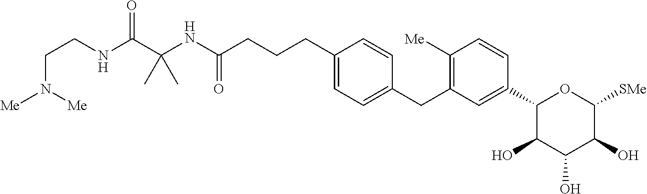 [SMILES: CS[C@H]1O[C@@H](c2ccc(C)c(Cc3ccc(CCCC(=O)NC(C)(C)C(=O)NCCN(C)C)cc3)c2)[C@H](O)[C@@H](O)[C@@H]1O]

and pharmaceutically acceptable salts and co-crystals thereof.

The discovery and preparation of LX2761-which exhibits very low systemic exposure in rodents and humans—have been disclosed. See, e.g., Goodwin, N.C., et al., Discovery of LX2761, a Sodium-dependent Glucose Cotransporter 1 (SGLT1) Inhibitor Restricted to the Intestinal Lumen, for the Treatment of Diabetes, J Med Chem. 2017 Jan. 26; 60(2):710-721; Powell, D. R., et al., LX2761, a Sodium/Glucose Cotransporter 1 Inhibitor Restricted to the Intestine, Improves Glycemic Control in Mice, J Pharmacol Exp Ther 2017 July; 362(1):85-97. Particular solid forms of LX2761 include amino acid co-crystals of the compound. A particular amino acid is L-proline. See U.S. Pat. No. 10,968,192.

This invention is based, in part, on the discovery that low doses of LX2761 can safely yet dramatically affect both the stool of both healthy, normal patients and those with type 2 diabetes. It is also based on the discovery that administration of the compound can lower cecal pH. It is believed that by causing a more acidic intestinal environment, administration of LX2761 can reduce the concentration of free ammonia in the gut, which in turn can reduce HE risk and/or severity. This combination of loose stool or diarrhea—which aid in emptying a patient's colon of toxic waste—and lower free ammonia may be particularly effective in reducing the severity of HE or even in preventing its occurrence. The fact that LX2761 has very low systemic exposure is a further advantage since patients with HE have compromised hepatic function. A further advantage is that while the administration of LX2761 can cause diarrhea in humans at low doses, its administration in the human clinical trial described below was generally not attended by severe cramping or other adverse effects associated with existing agents used to treat HE, such as lactulose.

Thus, one embodiment of this invention encompasses a method of treating or preventing HE, which method comprises administering to a patient in need thereof a therapeutically or prophylactically effective amount of a compound, wherein the compound is LX2761 or a pharmaceutically acceptable salt or co-crystal thereof. In certain embodiments, the compound is N-(1-((2-(dimethylamino)ethyl)-amino)-2-methyl-1-oxopropan-2-yl)-4-(4-(2-methyl-5-((2S,3R,4R,5S,6R)-3,4,5-trihydroxy-6-(methylthio)tetrahydro-2H-pyran-2-yl)benzyl)phenyl)butanamide L-proline. In particular embodiments, the compound is crystalline. One embodiment is directed to methods of treating HE; another is directed to methods of preventing HE (e.g., in patients predisposed to HE, such as those who previously had HE and those with cirrhosis).

In one embodiment of the invention, the patient is an adult human and the therapeutically or prophylactically effective amount is 0.025-1 mg (e.g., 0.25, 0.5, 0.1, 0.2, 0.3, 0.4, 0.5, 0.6 mg). In one embodiment, the compound is administered once daily (qd) or twice daily (bid).

In one embodiment, the HE is WHC grade I-III. In one embodiment, the HE is subclinical or minimal (e.g., WHC grade V). In one embodiment, the patient has type 2 diabetes mellitus.

In one embodiment, the patient is taking an antibiotic, lactulose/lactitol, LOLA, ornithine phenylacetate, glycerol/sodium phenylbutyrate, or zinc.

In some embodiments, the SGLT1 inhibitor (e.g., LX2761) is administered in combination with one or more additional agents that may be useful in treating HE or its underlying causes. Examples of such agents include: antibiotics (e.g., rifaximin, neomycin/paromomycin/metronidazole, vancomycin); lactulose/lactitol; LOLA (a preparation of L-ornithine and L-aspartate); ornithine phenylacetate; glycerol/sodium phenylbutyrate; and zinc (to correct underlying deficiency common in cirrhotic patients).

This invention also encompasses pharmaceutical compositions useful in the treatment, management, and/or prevention of HE. One embodiment encompasses a composition comprising a locally-acting SGLT1 inhibitor (e.g., LX2761 or a pharmaceutically acceptable salt or co-crystal thereof) and a second active agent useful in treating HE or its underlying causes, such as an antibiotic (e.g., rifaximin, neomycin/paromomycin/metronidazole, vancomycin), lactulose/lactitol, LOLA, ornithine phenylacetate, glycerol/sodium phenylbutyrate, or zinc.

Certain embodiments of the invention comprise testing of a patient for HE before administering an SGLT1 inhibitor. Tests currently used to diagnose HE and MHE include the Number Connection Tests (NCT) A and B, the Digit Symbol Test, the Block Design Test, the Portosystemic Encephalopathy Syndrome (PSE) Test providing the Psychometric Hepatic Encephalopathy Score (PHES), the Inhibitory Control Test, the Stroop Test, the Scan Test, the Continuous Reaction Time Test, the Cognitive Drug Research (CDR) test battery or the Repeatable Battery for the Assessment of Neuropsychological Status (RBANS). Weissenbom, supra, at S5. See also Rose, supra, at 1528. A preferred test is PHES.

### EXAMPLES

### Example 1. Study of LX2761 in Mice

This study evaluated the effect of orally administered LX2761 on glycemic control in preclinical models.

Mice and Rats. All studies were performed at Lexicon Pharmaceuticals, Inc., in strict accordance with the recommendations in the Guide for the Care and Use of Laboratory Animals of the National institutes of Health. The protocols for all studies were approved by the Lexicon Institutional Animal Care and Use Committee (OLAW Assurance Number A4152-01; AAALAC International Accreditation Number 001025). General methods for mouse and rat care have been described elsewhere. C57BL/6-Tyrc-Brd mice obtained from an in-house colony were fed either standard rodent chow diet (5010, LabDiet; PMI Nutrition International, St. Louis, MO), high-glucose diet (HGD; D12451; Research Diets, New Brunswick, NJ) with a kcal distribution of 20% from protein/10% from lard fat/35% from sucrose/35% from standard starch, HGD containing resistant starch 4 (HGD-RS4; D13072202; Research Diets) with a kcal distribution of 31% from protein/16% from lard fat/35% from sucrose/11% from standard starch/7% from RS4 (Fibersym RW, MGP, Inc., Atchison, KS), or HGD containing resistant starch 2 (HGD-RS2; D13072201; Research Diets) with a kcal distribution of 25% from protein/13% from lard fat/35% from sucrose/9% from standard starch/18% from RS2 (H-Maize 260; Honeyville, Inc., Salt Lake City, UT). Crl:CD Sprague-Dawley Rats (Charles River Laboratories International, Wilmington, MA) were obtained at 4 weeks of age and fed either chow or HGD. For all in vivo studies, mice and rats were fed HGD because glucose must be a major source of dietary calories if the effects of SGLT1 inhibition on glucose homeostasis are to be evaluated. All mice and rats were maintained in a temperature-controlled environment on a fixed 12-hour light/12-hour dark cycle and with free access to water and food. For each study: 1) mice or rats in each study group were taken from the same animal cohort and housed in the same room under comparable conditions; 2) mice and rats were randomly assigned a number that had no relation to body weight; and 3) mice and rats were randomized to individual study groups by body weight unless stated otherwise.

Oral Glucose Tolerance Tests. Oral glucose tolerance tests (OGTTs) were performed on unanesthetized adult male mice and rats. Mice: after pre-dose, blood samples were collected from the retro-orbital plexus at baseline, each mouse received, by oral gavage, 4 g glucose/kg body weight. Retro-orbital blood samples were then collected at multiple subsequent time points and assayed for whole-blood glucose. Rats: After pre-dose blood samples were collected from the saphenous vein at baseline, each rat received, by oral gavage, 4 g glucose/kg body weight. Saphenous blood samples were then collected at 10, 30, 60, and 120 minutes after glucose challenge and assayed for whole-blood glucose.

Glucose-Containing Meal Challenge. Adult male mice fed HGD for 6 days prior to study were randomized into two treatment groups. Vehicle or LX2761 was administered by oral gavage at 8:00 AM once daily for 4 consecutive days; body weight and food consumption were measured daily. On day 4, a meal consisting of HGD supplemented with glucose was prepared by adding 50 g of HGD powder and 9.4 g of glucose to H2O; the final volume was 94 ml, as described previously. The meal was prepared and kept at 60° C. until used. At 6 hours after the last dose of LX2761, the mice received 25 ml/kg of this meal (9.2 g/kg glucose, 2.5 g/kg protein, 0.6 g/kg fat) by oral gavage. Blood samples were collected by retro-orbital bleeding at 0, 10, 30, and 60 minutes after meal challenge for assessment of glucose excursion. In addition, blood samples collected 60 minutes after meal challenge were used to measure circulating levels of tGLP-1. Mice were necropsied at the end of the meal challenge (7 hours after the last dose of LX2761) and their cecal contents were collected and analyzed for pH and total glucose.

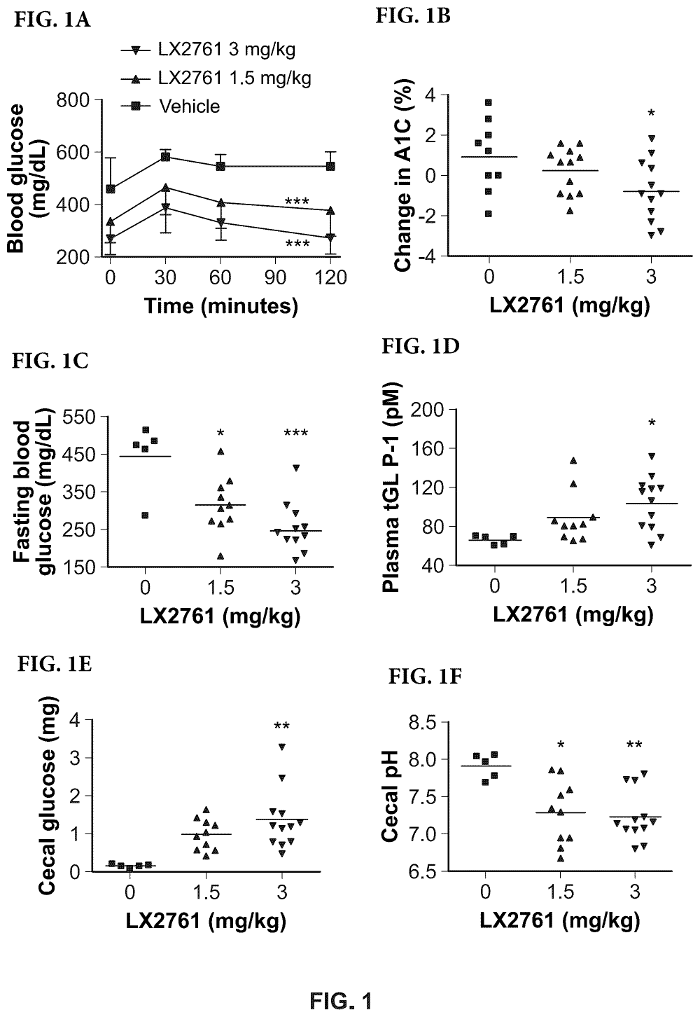

Results. In this study, adult male mice (12-13/group at randomization) with well-established diabetes (AIC>5.7% required for entry, mean=9.4% on day 1) received single daily doses of vehicle or LX2761 (1.5 or 3 mg/kg) by oral gavage. As shown in FIG. 1, administration of LX2761 improved glycemic control and reduced cecal pH (FIG. 1F).

### Example 2. Clinical Study of LX2761 in Humans

A phase 1 multiple ascending dose (MAD) clinical study of LX2761 in otherwise healthy subjects with type 2 diabetes (T2DM) was conducted, following an earlier, single ascending dose study of the compound's safety and tolerability.

This study evaluated the safety and tolerability of orally administered LX2761 given qd (i.e., once daily) or bid (i.e., twice daily) for eight days, over a range of ascending multiple dose levels in subjects with T2DM. As secondary objectives, the study evaluated the PD and PK of LX2761. Of the 51 subjects enrolled in the study, 27 (52.9%) were male and 24 (47.1%) were female. Subject ages ranged from 41 to 65 years, with BMIs ranging from 20.1 to 39.8 kg/m².

Subjects taking LX2761 were grouped into five cohorts: Cohort A=LX2761 dosed 0.4 mg qd for 4 days followed by LX2761 dosed 0.6 mg qd for 4 days; Cohort A2=LX2761 dosed 0.2 mg bid for 8 days; Cohort B=LX2761 dosed 0.6 mg qd for 4 days followed by LX2761 dosed 0.8 mg qd for 4 days; Cohort C=LX2761 dosed 0.6 mg qd for 4 days followed by LX2761 dosed 1.0 mg qd for 4 days; Cohort C1=LX2761 dosed 0.4 mg bid for 8 days.

No clinically significant changes were observed in clinical laboratory evaluations, vital signs, physical examination findings, or ECG parameters.

Pharmacodynamic results showed increases in peptide YY (PYY) and total glucagon-like peptide 1 (GLP-1) after LX2761 dosing, consistent with luminal glucose reaching the lower intestine. No statistically significant differences from placebo in the change from Baseline in urinary glucose excretion from 0 to 24 hours (UGE₀₋₂₄) or postprandial glucose (PPG) concentration area under the curve from 0 to 3 hours (AUC₀₋₃) were observed for any LX2761 cohort. However, least squares (LS) mean UGE₀₋₂₄ decreased from Baseline in all LX2761 cohorts at some time points while increasing from Baseline in the placebo cohort at each time point. The PPG concentration AUC₀₋₃ was reduced from Baseline in each LX2761 cohort, except Cohort A2, at most assessment time points. Overall, the PD data are consistent with a local effect at the SGLT1 transporter. These results suggest a mechanism of action for LX2761 whereby glucose absorption into the plasma is inhibited, causing glucose to remain in the digestive tract where it triggers secretion of PYY and GLP-1 from enteroendocrine L cells, predominately in the distal/small intestine.

The pharmacokinetics (PK) of LX2761 showed rapid absorption, as expected following administration of an oral solution. The exposure was limited as shown by mostly BLQ concentrations after the first dose. The increase in exposure (AUC and Cₘₐₓ) was generally dose-related and associated with moderate to high variability.

The percentage of subjects reporting any TEAE (treatment-emergent adverse events) in the LX2761 cohorts ranged from 22.2% to 37.5% and was 10.0% in the placebo cohort. All reported TEAEs were of mild or moderate intensity. No severe TEAEs, SAEs (serious adverse events), deaths, or discontinuations due to TEAEs occurred. The incidence of subjects reporting any TEAE was identical in the LX2761 qd and bid cohorts that received similar total doses.

The most frequently reported TEAEs were gastrointestinal (GI) events and consisted mainly of nausea, vomiting, and abdominal pain. The type, severity, and frequency of GI TEAEs observed for LX2761 in this study were consistent with that expected for an SGLT1-selective inhibitor. Gastrointestinal events were reported more frequently in the higher dose level cohorts (C and C1), although the small number of subjects in each cohort does not allow robust comparison between the cohorts.

Reporting of TEAEs of diarrhea was not based on subject self-reporting. Given the mechanism of action of LX2761, osmotic diarrhea was expected to occur in all subjects receiving LX2761 based on either stool form or daily frequency. Hence diarrhea was not captured as a TEAE; instead, every bowel movement (BM) was assessed and recorded by study personnel using daily frequency of BMs and Bristol Stool Form (BSF) Scale. The study stopping rules defined gradation of diarrhea as follows: diarrhea of moderate intensity was defined as an increase of 4 to 5 stools/day over the normal pre-study count with moderate cramping that limits usual daily activity or may require simple therapeutic intervention; diarrhea of severe intensity was defined as an increase of 6 to 8 stools/day over the normal pre-study count with severe cramping or incontinence that prohibits usual daily activities or requires systemic drug therapy or other therapeutic intervention. No change in BMs meeting these criteria of moderate or severe diarrhea was reported in this study.

All 25 TEAEs were of mild or moderate intensity. Of these, 9 TEAEs were assessed as moderate in intensity, all reported by subjects (n=6) treated with LX2761. The remaining 16 TEAEs were of mild intensity.

All but one of the 25 TEAEs were considered by the Investigator to be possibly, probably, or definitely related to study treatment, including all nine TEAEs of moderate intensity: one event of nausea and 1 event of abdominal pain in different subjects in Cohort A; one event of vomiting in one subject, and one event of vomiting and one event of syncope by a second subject in Cohort C; and one event of alanine aminotransferase (ALT) increased and one event of aspartate aminotransferase (AST) increased in one subject, and one event of nausea and one event of flatulence in a second subject in Cohort C1. All TEAEs, regardless of intensity, resolved by the end of the study.

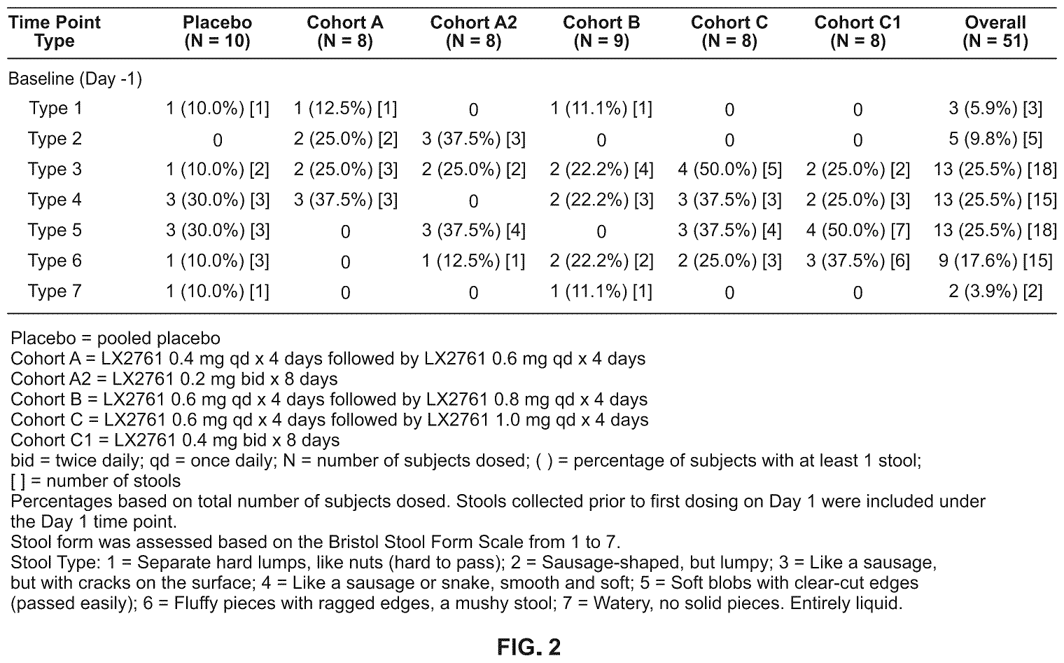

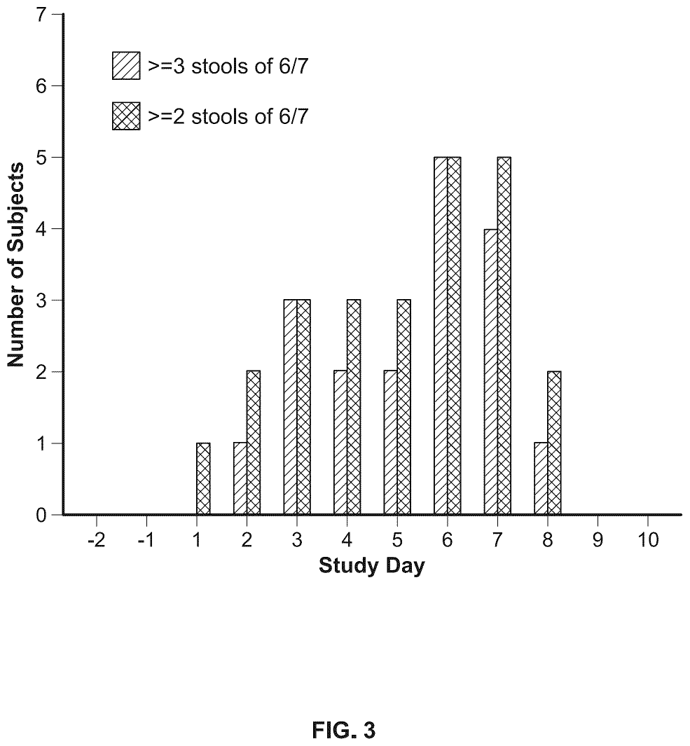

As shown in FIG. 2, subjects' mean stool frequency per day increased by >0.5 BM from Baseline in all LX2761 cohorts on at least one day through Day 8; in placebo subjects, there was minimal change in mean stool frequency per day. Shifts to BSF Scale Type 6 or 7 occurred in placebo subjects and all LX2761 cohorts, and in all but Cohort B of LX2761 cohorts, 50% or more of subjects had BMs graded as BSF Scale Type 6 or 7 on at least one day through Day 8. The highest percentage of subjects with BMs graded as BSF Scale Type 6 or 7 on a single treatment day in placebo subjects was 30%. The frequency of subjects with 2 or 3 BMs graded as BSF Scale Type 6 or 7 on a single day through Day 8 was higher in all LX2761 cohorts than in placebo-treated subjects. FIG. 3 shows the number of BMs graded 6 or 7 in Cohort 2A as a function of time.

In sum, the TEAEs reported in subjects with T2DM who received LX2761 were consistent with those expected in an SGLT1-selective inhibitor and consisted mainly of GI events. The reported TEAEs were generally mild, and none led to discontinuation of LX2761. As expected, based on SGLT1 inhibition, greater increases in BM frequency and changes in BSF Scale scores were observed in subjects treated with LX2761 than in placebo-treated subjects. No subject experienced any change in BMs that met the study definition of moderate or severe diarrhea, and none of the stopping criteria related to diarrhea were met.

All publications (e.g., patents and patent applications) cited above are incorporated herein by reference in their entireties.

1. A method of treating or preventing hepatic encephalopathy (HE), which method comprises administering to a patient in need thereof a therapeutically or prophylactically effective amount of a compound, which compound inhibits SLGT1 and has minimal systemic exposure.

2. The method of claim 1, wherein the compound is N-(1-((2-(dimethylamino)ethyl)-amino)-2-methyl-1-oxopropan-2-yl)-4-(4-(2-methyl-5-((2S,3R,4R,5S,6R)-3,4,5-trihydroxy-6-(methylthio)tetrahydro-2H-pyran-2-yl)benzyl)phenyl)butanamide (LX2761): 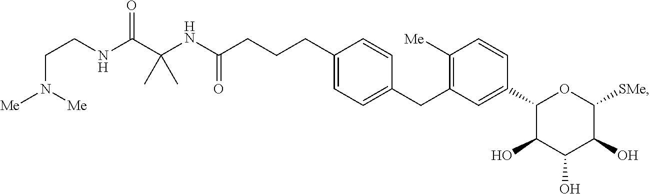 [SMILES: CS[C@H]1O[C@@H](c2ccc(C)c(Cc3ccc(CCCC(=O)NC(C)(C)C(=O)NCCN(C)C)cc3)c2)[C@H](O)[C@@H](O)[C@@H]1O]
or a pharmaceutically acceptable salt or co-crystal thereof.

3. The method of claim 1, wherein the patient is at risk of having HE.

4. The method of claim 2, wherein the compound is N-(1-((2-(dimethylamino)ethyl)-amino)-2-methyl-1-oxopropan-2-yl)-4-(4-(2-methyl-5-((2S,3R,4R,5S,6R)-3,4,5-trihydroxy-6-(methylthio)tetrahydro-2H-pyran-2-yl)benzyl)phenyl)butanamide L-proline.

5. The method of claim 4, wherein the compound is crystalline.

6. The method of claim 1, wherein the patient has type 2 diabetes mellitus.

7. The method of claim 2, wherein the therapeutically or prophylactically effective amount is 0.025-1 mg.

8. The method of claim 7, wherein the compound is administered once or twice daily (bid).

9. The method of claim 1, wherein the HE is WHC grade I-III.

10. The method of claim 9, wherein the HE is subclinical or minimal hepatic encephalopathy.

11. The method of claim 1, wherein the patient is taking an antibiotic, lactulose/lactitol, LOLA, ornithine phenylacetate, glycerol/sodium phenylbutyrate, or zinc.

12. A composition comprising a compound and a second active agent, wherein the compound is an SGLT1 inhibitor with minimal systemic exposure and the second active agent is an antibiotic, lactulose/lactitol, LOLA, ornithine phenylacetate, glycerol/sodium phenylbutyrate, or zinc.

13. The composition of claim 12, wherein the compound is LX2761 or a pharmaceutically acceptable salt or co-crystal thereof.

14. The composition of claim 13, wherein the compound is N-(1-((2-(dimethylamino)ethyl)-amino)-2-methyl-1-oxopropan-2-yl)-4-(4-(2-methyl-5-((2S,3R,4R,5S,6R)-3,4,5-trihydroxy-6-(methylthio)tetrahydro-2H-pyran-2-yl)benzyl)phenyl)butanamide L-proline.

15. The composition of claim 14, wherein the compound is crystalline.

16. The composition of claim 12, wherein the second active agent is lactulose/lactitol.
